(19) United States
(12) Patent Application Publication
(10) Pub. No.: US 2018/0228921 A1
(43) Pub. Date: Aug. 16, 2018

(54) VISUALIZING VIRAL RESERVOIRS AND DISSEMINATION IN VIVO

(71) Applicant: EXCISION BIOTHERAPEUTICS, INC., Bedminster, NJ (US)

(72) Inventors: Kamel Khalili, Bala Cynwyd, PA (US); Won-Bin Young, Pittsburgh, PA (US)

(73) Assignee: EXCISION BIOTHERAPEUTICS, INC., Bedminster, NJ (US)

(21) Appl. No.: 15/895,414

(22) Filed: Feb. 13, 2018

Related U.S. Application Data
(60) Provisional application No. 62/458,185, filed on Feb. 13, 2017.

(51) Int. Cl. A61K 49/00; A61K 38/46; A61K 31/7088; A61P 31/18

(52) U.S. Cl. CPC A61K 49/0013; A61K 38/465; A61K 48/00; A61P 31/18; A61K 31/7088

(57) ABSTRACT

A reporter gene that can be administered to an individual such as HIV-BAL-eLuc in order to detect the presence of a virus. A method of detecting the presence of virus in an individual, by administering a reporter gene to the individual, associating the reporter gene with the virus, imaging the individual, and detecting the presence of virus in the individual. A method of determining the efficacy of a treatment for a virus, by administering a reporter gene to the individual receiving treatment for the virus, associating the reporter gene with the virus, imaging the individual, detecting the presence of virus in the individual, and determining if the treatment is effective.

### BACKGROUND OF THE INVENTION

### 1. Technical Field

The present invention relates to methods and compositions for in vivo imaging. More specifically, the present invention relates to methods and compositions for detecting viruses in vivo.

### 2. Background Art

For more than three decades since the discovery of HIV-1, AIDS remains a major public health problem affecting greater than 35.3 million people worldwide. AIDS remains incurable due to the permanent integration of HIV-1 into the host genome. Current therapy (highly active antiretroviral therapy or HAART) for controlling HIV-1 infection and impeding AIDS development profoundly reduces viral replication in cells that support HIV-1 infection and reduces plasma viremia to a minimal level. But HAART fails to suppress low level viral genome expression and replication in tissues and fails to target the latently-infected cells, for example, resting memory T cells, brain macrophages, microglia, and astrocytes, gut-associated lymphoid cells, that serve as a reservoir for HIV-1. Persistent HIV-1 infection is also linked to co-morbidities including heart and renal diseases, osteopenia, and neurological disorders.

Persistence of HIV-1 reservoirs greatly impedes a possible cure even during suppressive antiretroviral therapy (ART). Current approaches of monitoring viral levels are based on tissue biopsy and biomarker from blood samples to measure the viral load for evaluating the effectiveness of drug treatment. These approaches are prone to sampling bias and error. In some tissues and organs, such as the brain, repeated biopsy is unlikely. The results from the conventional approaches are obtained with only portion of the tissues which only partially represent the outcomes.

Emory University has developed an imaging method of HIV anatomical reservoir locations for monitoring of HIV. Combined positron emission tomography (PET) and computer tomography (CT) is used to visualize a radiolabelled gp120 antibody that recognizes HIV virions and infected cells. This method provides whole body views of viral replication, detection of reservoirs of residual replication during therapy, viral resurgence, and response to antiviral treatments with high sensitivity. There are drawbacks to this method. This approach can only detect HIV-infected cells with active expression of viral proteins. In fact, most of the HIV and SIV reservoirs they are in latent stage (silence) and do not express viral proteins and would therefore go undetected.

There remains a need for an effective method of detecting viruses and reservoirs of viruses within the body.

### SUMMARY OF THE INVENTION

The present invention provides for a reporter gene that can be administered to an individual such as HIV-BAL-eLuc in order to detect the presence of a virus.

The present invention provides for a method of detecting the presence of virus in an individual, by administering a reporter gene to the individual, associating the reporter gene with the virus, imaging the individual, and detecting the presence of virus in the individual.

The present invention also provides for a method of determining the efficacy of a treatment for a virus, by administering a reporter gene to the individual receiving treatment for the virus, associating the reporter gene with the virus, imaging the individual, detecting the presence of virus in the individual, and determining if the treatment is effective.

The present invention is useful for screening drugs against infection, in this case, HIV, rather than requiring the process of detecting a virus. For example, the reporter virus and in vivo imaging can be used as readout for the efficacy of drugs and vaccines.

### DESCRIPTION OF THE DRAWINGS

Other advantages of the present invention are readily appreciated as the same becomes better understood by reference to the following detailed description when considered in connection with the accompanying drawings wherein:

FIGS. 1A-1H show photographs of luciferase signal in mice: FIG. 1A at day 3, FIG. 1B at day 7, FIG. 1C at day 14, FIG. 1D at day 21, FIG. 1E at day 28, FIG. 1F at day 35, FIG. 1G at day 42, and FIG. 1H at day 63.

### DETAILED DESCRIPTION OF THE INVENTION

The present invention is generally directed to methods and compositions for imaging the presence of virus and viral reservoirs in the body of an individual. The invention can be a monitor for gene editing therapy. For example, it can monitor a patient being given a gene editing therapeutic and determine sufficient dosing based on viral pool eradication. As a readout of gene editing on HIV, eradication is one of the applications. The invention can also be used for screening a library of drugs and vaccines. The imaging intensity can be digitalized and the locations of drug resistance or sensitivity and kinetics can be recorded in real time.

False positives from other viruses are highly unlikely. The reporter gene is genetically engineered with the specific target viral genome. Only the specific target virus genome can be detected because of this strategy.

“Lysogenic virus” as used herein, refers to a virus that replicates by the lysogenic cycle (i.e. does not cause the host cell to burst and integrates viral nucleic acid into the host cell DNA). The lysogenic virus can mainly replicate by the lysogenic cycle but sometimes replicate by the lytic cycle. In the lysogenic cycle, virion DNA is integrated into the host cell, and when the host cell reproduces, the virion DNA is copied into the resulting cells from cell division. In the lysogenic cycle, the host cell does not burst.

“Lytic virus” as used herein refers to a virus that replicates by the lytic cycle (i.e. causes the host cell to burst after an accumulation of virus within the cell). The lytic virus can mainly replicate by the lytic cycle but sometimes replicate by the lysogenic cycle.

The present invention provides for a composition of a reporter gene that can be administered to an individual such as HIV-BAL-eLuc in order to detect the presence of a virus. Using a reporter gene and in vivo imaging allows for whole body scans for unknown HIV reservoirs and to monitor where and when HIV rebound occurs. This approach can also be used to measure the penetration of vaccine, antiretroviral drugs, and immunotherapies into different tissues and organs based on the imaging as readout for the therapeutic efficacy of the tested reagents.

The reporter gene is genetically engineered into a viral genome. For example using HIV, once the reporter gene is engineered into the HIV and the HIV is infected, the expression of the reporter gene is dependent on HIV gene expression, thus the presence of reporter gene expression can represent HIV infection and its activity in real time. HIV inoculation can be done by intravaginal administration to recapitulate the transmitting route in humans.

Progress of the treatment can be a drop in number of virions in the body or eradication of the virus in the body. The virus can be, but is not limited to, a lysogenic virus or a lytic virus, or a virus that has both lysogenic and lytic properties, such as, but not limited to any virus in the following TABLES 1-12.

TABLE 1 lists viruses in the picornaviridae/hepeviridae/flaviviridae families and their method of replication.

TABLE 1

| | | |
|---|---|---|
| Hepatitis A | +ssRNA viral genome | Lytic/Lysogenic Replication |
| | | cycle |
| Hepatitis B | dsDNA-RT viral genome | Lysogenic Replication cycle |
| Hepatitis C | +ssRNA viral genome | Lytic Replication cycle |
| Hepatitis D | −ssRNA viral genome | Lytic/Lysogenic Replication |
| Hepatitis E | +ssRNA viral genome | cycle |
| Coxsachievirus | | Lytic Replication cycle |

TABLE 2 lists viruses in the herpesviridae family and their method of replication.

TABLE 2

| | | |
|---|---|---|
| HSV-1 (HHV1) | dsDNA viral genome | Lytic/Lysogenic |
| | | Replication cycle |
| HSV-2 (HHV2) | dsDNA viral genome | Lytic/Lysogenic |
| | | Replication cycle |
| Cytomegalovirus (HHV5) | dsDNA viral genome | Lytic/Lysogenic |
| | | Replication cycle |
| Epstein-Barr Virus (HHV4) | dsDNA viral genome | Lytic/Lysogenic |
| | | Replication cycle |
| Varicella Zoster Virus | dsDNA viral genome | Lytic/Lysogenic |
| (HHV3) | | Replication cycle |
| Roseolovirus (HHV6A/B) | | |
| HHV7 | | |
| HHV8 | | |

TABLE 3 lists viruses in the orthomyxoviridae family and their method of replication.

TABLE 3

| | |
|---|---|
| Influenza Types A, B, C, D | −ssRNA viral genome |

TABLE 4 lists viruses in the retroviridae family and their method of replication.

TABLE 4

| | | |
|---|---|---|
| HIV1 and HIV2 | +ssRNA viral | Lytic/Lysogenic Replication cycle |
| | genome | |
| HTLV1 and HTLV2 | +ssRNA viral | Lytic/Lysogenic Replication cycle |
| | genome | |
| Rous Sarcoma Virus | +ssRNA viral | Lytic/Lysogenic Replication cycle |
| | genome | |

TABLE 5 lists viruses in the papillomaviridae family and their method of replication.

TABLE 5

| | | |
|---|---|---|
| HPV family | dsDNA viral genome | Budding from desquamating cells |
| | | (semi-lysogenic) |

TABLE 6 lists viruses in the flaviviridae family and their method of replication.

TABLE 6

| | | |
|---|---|---|
| Yellow Fever | +ssRNA viral | Budding/Lysogenic Replication |
| | genome | |
| Zika | +ssRNA viral | Budding/Lysogenic Replication |
| | genome | |
| Dengue | +ssRNA viral | Budding/Lysogenic Replication |
| | genome | |
| West Nile | +ssRNA viral | Budding/Lysogenic Replication |
| | genome | |
| Japanese Encephalitis | +ssRNA viral | Budding/Lysogenic Replication |
| | genome | |

TABLE 7 lists viruses in the reoviridae family and their method of replication.

TABLE 7

| | | |
|---|---|---|
| Rota | dsRNA viral genome | Lytic Replication cycle |
| Seadornvirus | dsRNA viral genome | Lytic Replication cycle |
| Coltivirus | dsRNA viral genome | Lytic Replication cycle |

TABLE 8 lists viruses in the rhabdoviridae family and their method of replication.

TABLE 8

| | | |
|---|---|---|
| Lyssa Virus (Rabies) | −ssRNA viral | Budding/Lysogenic Replication |
| | genome | |
| Vesiculovirus | −ssRNA viral | Budding/Lysogenic Replication |
| | genome | |
| Cytorhabdovirus | −ssRNA viral | Budding/Lysogenic Replication |
| | genome | |

TABLE 9 lists viruses in the bunyanviridae family and their method of replication.

TABLE 9

| | | |
|---|---|---|
| Hantaan Virus | tripartite −ssRNA viral genome | Budding/Lysogenic |
| | | Replication |
| Rift Valley Fever | tripartite −ssRNA viral genome | Budding/Lysogenic |
| | | Replication |
| Bunyamwera | tripartite −ssRNA viral genome | Budding/Lysogenic |
| Virus | | Replication |

TABLE 10 lists viruses in the arenaviridae family and their method of replication.

TABLE 10

| | | |
|---|---|---|
| Lassa Virus | ssRNA viral genome | Budding/Lysogenic Replication |
| Junin Virus | ssRNA viral genome | Budding/Lysogenic Replication |
| Machupo Virus | ssRNA viral genome | Budding/Lysogenic Replication |
| Sabia Virus | ssRNA viral genome | Budding/Lysogenic Replication |
| Tacaribe Virus | ssRNA viral genome | Budding/Lysogenic Replication |
| Flexal Virus | ssRNA viral genome | Budding/Lysogenic Replication |
| Whitewater | ssRNA viral genome | Budding/Lysogenic Replication |
| Arroyo | | |
| Virus | | |

TABLE 11 lists viruses in the filoviridae family and their method of replication.

TABLE 11

| | | |
|---|---|---|
| Ebola | RNA viral genome | Budding/Lysogenic Replication |
| Marburg Virus | RNA viral genome | Budding/Lysogenic Replication |

TABLE 12 lists viruses in the polyomaviridae family and their method of replication.

TABLE 12

| | | |
|---|---|---|
| JC Virus | dsDNA circular viral genome | Lytic/Lysogenic Replication |
| | | cycle |
| BK Virus | dsDNA circular viral genome | Lytic/Lysogenic Replication |
| | | cycle |

The treatment for the virus can be ART agents for HIV, such as, but not limited to, reverse transcriptase inhibitors (e.g., nucleoside/nucleotide reverse transcriptase inhibitors, zidovudine, emtricitibine, lamivudine and tenofivir; and non-nucleoside reverse transcriptase inhibitors such as efavarenz, nevirapine, rilpivirine); protease inhibitors, e.g., tipiravir, darunavir, indinavir; entry inhibitors, e.g., maraviroc; fusion inhibitors, e.g., enfuviritide; or integrase inhibitors e.g., raltegrivir, dolutegravir. Exemplary ART agents can also include multi-class combination agents for example, combinations of emtricitabine, efavarenz, and tenofivir; combinations of emtricitabine; rilpivirine, and tenofivir; or combinations of elvitegravir, cobicistat, emtricitabine and tenofivir.

The treatment for the virus can also be vectors encoding various gene editors that target and excise or otherwise render the virus inoperable such as CRISPR-associated nucleases such as Cas9, Cpf1, C2c1, C2c3, TevCas9, Archaea Cas9, CasY, and CasX gRNAs, Argonaute endonuclease gDNAs and other gene editors that target viral DNA. In general, gene editing allows DNA or RNA to be inserted, deleted, or replaced in an organism's genome by the use of nucleases. Preferably, an entire genome of the virus is excised from the individual's cells. Without using in vivo imaging, in general, the presence of virus in human body can only be measured from the blood without knowing the viral burden in each solid tissue.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a method of detecting the presence of virus in an individual, by administering a reporter gene to the individual, associating the reporter gene with the virus, imaging the individual, and detecting the presence of virus in the individual. The virus can be any of those described above. Preferably, the reporter gene is administered by injection. The imaging can be any in vivo imaging technique that can detect the reporter gene. If virus is detected by the method, any of the treatments described above can be administered to the individual. The reporter gene can be changed or modified to ones designated for other imaging approaches, such as magnetic resonance imaging (MRI) or positron emission tomography (PET). These imaging approaches are routine in clinical and the bioluminescence imaging (BLI) and presently has only be used in animal models.

The present invention also provides for a method of determining the efficacy of a treatment for a virus, by administering a reporter gene to the individual receiving treatment for the virus, associating the reporter gene with the virus, imaging the individual, detecting the presence of virus in the individual, and determining if the treatment is effective. Depending on the results, if virus remains in the body and is detected, dosing of the treatment can be adjusted or a different treatment can be prescribed. This method can be performed at various time points during treatment to determine the progress of the treatment. The virus can be any of those described above. Preferably, the reporter gene is administered by injection. The imaging can be any in vivo imaging technique that can detect the reporter gene. As discussed above the reporter gene can be changed or modified to ones designated for other imaging approaches, such as magnetic resonance imaging (MRI) or positron emission tomography (PET). These imaging approaches are routine in clinical and the bioluminescence imaging (BLI) can only be used in animal models.

The present invention has several advantages. Non-invasive in vivo imaging can reveal the uncharacterized anatomic and pharmacological sanctuaries of deep tissues for isolating latently infected cells and drug-resistant HIV-1 mutants during suppressive antiretroviral treatment. This is particularly useful for studying tissues in which obtaining biopsies is not feasible, such as the brain, or where only a few infected cells can be obtained by a small biopsy, such as in the female genital tract. Most of all, it allows longitudinal observation on the same animals for long term effectiveness of therapeutic treatments without potential variation between subjects, which is urgently needed for identifying HIV reservoirs during suppressive antiretroviral therapy (ART). In vivo imaging can serve as a readout in real time to evaluate the effectiveness of suppressive ART, Pre-Exposure Prophylaxis (PrEP), or Post-Exposure Prophylaxis (PEP) antiretroviral treatment and to reveal where and when the viremia rebound.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

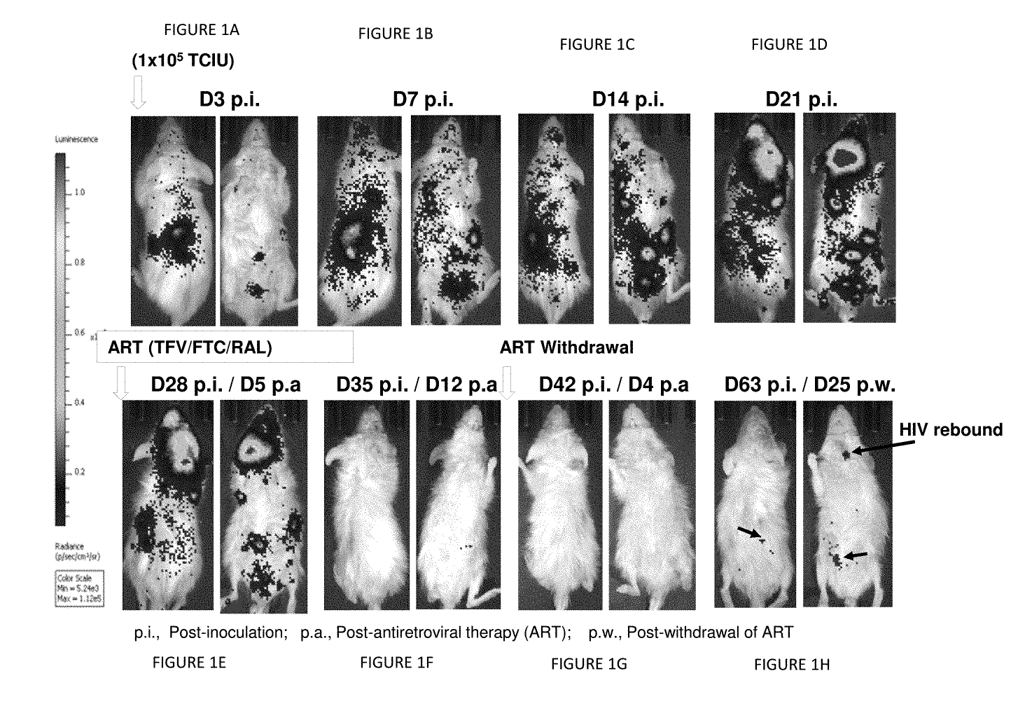

B10M4 mice injected with 1×10⁶ TCIU of HIV-BAL-eLuc show an increase in luciferase signal indicating rapid viral replication and spread over 21 day period post injection, as shown in FIGS. 1A-1H. After 28 days the mice are given ART inhibitors (TRUVADA® (emtricitabine and tenofovir disoproxil fumarate, Gilead Sciences) alone or with raltegravir). Specifically, Tenofovir disoproxil fumarate (TDF, 146 mg/kg bodyweight), Emtricitabine (FTC, 140 mg/kg bodyweight), and integrase inhibitor Raltegravir (RAL, 56 mg/kg bodyweight). These drugs were given to mice orally by mixing them into their diet.

These drugs were given to mice orally by mixing them into their diet. After 12 days post ART, the viral replication has been inhibited to levels of no detection. After this time point, the virus begins to rebound and becomes visible through luciferase reporter signal 25 days post withdrawal from ART.

The significance of this experiment is two fold. First, it represents an excellent model for testing EBT101 (CRISPR-Cas9 system) against ART withdrawal—‘weaning’. Second, virus rebounds after 25 days. In the previous figure, no viral rebound was observed after 93 days post EBT101 treatment.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition for detecting the presence of a virus, comprising a reporter gene.

2. The composition of claim 1, wherein said reporter gene is HIV-BAL-eLuc.

3. The composition of claim 1, wherein said virus detected is chosen from the group consisting of lytic viruses, lysogenic viruses, and combinations thereof.

4. The composition of claim 1, wherein said virus detected is chosen from the group consisting of hepatitis A, hepatitis B, hepatitis C, hepatitis D, hepatitis E, coxsachievirus, HSV-1, HSV-2, cytomegalovirus, Epstein-Barr virus, Varicella Zoster virus, roseolovirus, HHV7, HHV8, influenza type A, influenza type B, influenza type C, influenza type D, HIV1, HIV2, HTLV1, HTLV2, Rous sarcoma virus, HPV virus, yellow fever, zika virus, dengue, west nile virus, Japanese encephalitis, rota virus, seadornvirus, coltivirus, lyssa virus, vesiculovirus, cytorhabdovirus, Hantaan virus, Rift Valley fever, Bunyamwera virus, Lassa virus, Junic virus, Machupa virus, Sabia virus, Tacaribe virus, Flexal virus, Whitewater Arroyo virus, Ebola virus, Marburg virus, JV virus, and BK virus.

5. A method of detecting the presence of virus in an individual, including the steps of:
administering a reporter gene to the individual;
associating the reporter gene with the virus;
imaging the individual; and
detecting the presence of virus in the individual.

6. The method of claim 5, wherein the reporter gene is HIV-BAL-eLuc.

7. The method of claim 5, wherein said administering step is further defined as administering the reporter gene by injection to the individual.

8. The method of claim 5, wherein the virus is chosen from the group consisting of lytic viruses, lysogenic viruses, and combinations thereof.

9. The method of claim 5, wherein the virus is chosen from the group consisting of hepatitis A, hepatitis B, hepatitis C, hepatitis D, hepatitis E, coxsachievirus, HSV-1, HSV-2, cytomegalovirus, Epstein-Barr virus, Varicella Zoster virus, roseolovirus, HHV7, HHV8, influenza type A, influenza type B, influenza type C, influenza type D, HIV1, HIV2, HTLV1, HTLV2, Rous sarcoma virus, HPV virus, yellow fever, zika virus, dengue, west nile virus, Japanese encephalitis, rota virus, seadornvirus, coltivirus, lyssa virus, vesiculovirus, cytorhabdovirus, Hantaan virus, Rift Valley fever, Bunyamwera virus, Lassa virus, Junic virus, Machupa virus, Sabia virus, Tacaribe virus, Flexal virus, Whitewater Arroyo virus, Ebola virus, Marburg virus, JV virus, and BK virus.

10. The method of claim 5, wherein said detecting step is further defined as detecting latently infected cells in the individual.

11. The method of claim 5, further including, if virus is detected in said detecting step, the step of administering treatment to the individual.

12. The method of claim 11, wherein the treatment is chosen from the group consisting of reverse transcriptase inhibitors, protease inhibitors, entry inhibitors, fusion inhibitors, and integrase inhibitors.

13. The method of claim 11, wherein the treatment is a vector encoding a gene editor that targets the virus.

14. The method of claim 13, wherein the gene editor is chosen from the group consisting of Cas9 gRNAs, Cpf1 gRNAs, C2c1 gRNAs, C2c3 gRNAs, TevCas9 gRNAs, Archaea Cas9 gRNAs, CasY gRNAs, and CasX gRNAs, and Argonaute endonuclease gDNAs.

15. The method of claim 14, wherein the gene editor treats the virus by excising an entire genome of the virus.

16. A method of determining the efficacy of a treatment for a virus, including the steps of:
administering a reporter gene to the individual receiving treatment for the virus;
associating the reporter gene with the virus;
imaging the individual;
detecting the presence of virus in the individual; and
determining if the treatment is effective.

17. The method of claim 16, wherein the reporter gene is HIV-BAL-eLuc.

18. The method of claim 16, wherein said administering step is further defined as administering the reporter gene by injection to the individual.

19. The method of claim 16, wherein the virus is chosen from the group consisting of lytic viruses, lysogenic viruses, and combinations thereof.

20. The method of claim 16, wherein the virus is chosen from the group consisting of hepatitis A, hepatitis B, hepatitis C, hepatitis D, hepatitis E, coxsachievirus, HSV-1, HSV-2, cytomegalovirus, Epstein-Barr virus, Varicella Zoster virus, roseolovirus, HHV7, HHV8, influenza type A, influenza type B, influenza type C, influenza type D, HIV1, HIV2, HTLV1, HTLV2, Rous sarcoma virus, HPV virus, yellow fever, zika virus, dengue, west nile virus, Japanese encephalitis, rota virus, seadornvirus, coltivirus, lyssa virus, vesiculovirus, cytorhabdovirus, Hantaan virus, Rift Valley fever, Bunyamwera virus, Lassa virus, Junic virus, Machupa virus, Sabia virus, Tacaribe virus, Flexal virus, Whitewater Arroyo virus, Ebola virus, Marburg virus, JV virus, and BK virus.

21. The method of claim 16, wherein the treatment is chosen from the group consisting of reverse transcriptase inhibitors, protease inhibitors, entry inhibitors, fusion inhibitors, and integrase inhibitors.

22. The method of claim 16, wherein the treatment is a vector encoding a gene editor that targets the virus.

23. The method of claim 22, wherein the gene editor is chosen from the group consisting of Cas9 gRNAs, Cpf1 gRNAs, C2c1 gRNAs, C2c3 gRNAs, TevCas9 gRNAs, Archaea Cas9 gRNAs, CasY gRNAs, and CasX gRNAs, and Argonaute endonuclease gDNAs

24. The method of claim 22, wherein the gene editor treats the virus by excising an entire genome of the virus.

25. The method of claim 16, wherein if virus is detected in said detecting step, further including the step of adjusting treatment dosing.

26. The method of claim 16, wherein if virus is detected in said detecting step, further including the step of prescribing a different treatment.
